(19) United States
(12) Patent Application Publication
(10) Pub. No.: US 2009/0171323 A1
(43) Pub. Date: Jul. 2, 2009

(54) Catheter assembly and fluid supply apparatus therefor

(75) Inventors: Tyson L. Rugenstein, Camby, IN (US); Michael R. Kurrus, Ellettsville, IN (US); David A. Drewes, JR., Bloomington, IN (US); Gregory A. Frankland, Unionville, IN (US)

Correspondence Address: BRINKS HOFER GILSON & LIONE/CHICAGO/COOK, PO BOX 10395, CHICAGO, IL, 60610, US

(73) Assignees: COOK INCORPORATED, Bloomington, IN (US); SABIN CORPORATION, Bloomington, IN (US)

(21) Appl. No.: 12/005,177

(22) Filed: Dec. 26, 2007

(51) Int. Cl. A61M 25/18

(52) U.S. Cl. 604/537

(57) ABSTRACT

A catheter assembly includes a multi-lumen catheter and fluid supply apparatus for supplying fluid to said multi-lumen catheter. The fluid supply apparatus includes an inlet element for coupling to a supply of catheter fluid, a plurality of fluid outlet elements arranged to be each coupled to a respective lumen of the catheter and a flow splitter arranged between the inlet element and the outlet elements. The flow splitter is able to split or divert flow of fluid from the inlet to at least one of the plurality of outlets. For this purpose, the preferred embodiment includes a gate for directing fluid flow to one or more of the outlet elements. There may also be provided a diffuser and a nozzle.

### FIELD OF THE INVENTION

The present invention relates to a catheter assembly suitable for computer tomography (CT) scanning and to fluid supply apparatus for such a catheter assembly.

### BACKGROUND OF THE INVENTION

In some CT scan procedures it is desirable to enhance the scanned images of a patient for diagnostic purposes. Enhanced CT scans of this type are typically achieved by injecting into the patient, at the site to be scanned, a contrast agent or dye, which in some instances is iodine based. The injection of such a contrast agent is usually achieved by means of a catheter inserted intraluminally into the patient. The catheter typically is provided with a plurality of lumens extending the entire length of the catheter, one or more of these lumens being used for the supply of the contrast agent, from a source outside the patient, to the distal end of the catheter, which is in use located at the site to be diagnosed. The catheter lumens may also provide for a guide wire for use during insertion of the catheter into the patient and for other known purposes.

For enhanced CT scan procedures, the amount of contrast medium injected and the rate of injection of the medium can often affect the quality of the scan. As a result of this, it is often necessary to inject contrast media through the catheter at a specific flow rate or at least above a minimum specified flow rate.

A problem occurs with existing systems in that the limited diameter of lumen of the catheter provided for the contrast medium limits the rate of flow of contrast media injected therethrough. Increasing the diameter of the lumen is not always possible when multiple lumens are required in the catheter and also when the outer dimensions of the catheter are restricted as a result of the medical application. Increasing the flow pressure can lead to distortion of the catheter as well as possible rupture thereof, of the catheter or failure of one or more of the components of the assembly.

### SUMMARY OF THE PRESENT INVENTION

The present invention seeks to provide an improved catheter assembly suitable for computer tomography (CT) scanning and to improved fluid supply apparatus for such a catheter assembly.

According to an aspect of the present invention, there is provided fluid supply apparatus for supplying fluid to a multi-lumen catheter including an inlet element for coupling to a supply of catheter fluid, a plurality of fluid outlet elements and a flow splitter arranged between the inlet element and the outlet elements and able to split or divert flow of fluid from the inlet to at least one of the plurality of outlets.

In practice, the fluid outlet elements are each coupled to one of a plurality of lumens of a catheter. Thus, instead of fluid being supplied to only one of the lumens of the catheter, fluid can be supplied to a plurality of these, and thus through an overall wider cross-sectional luminal area. In some instances, the device can be used to supply fluid through lumens used also for other purposes, such as for a guide wire.

In the preferred embodiment, the flow splitter is configurable so as to adjust the flow to the outlet elements and thus through a catheter attached thereto. For example, the flow splitter may be settable to direct fluid to only one of the outlet elements or to both. In this example, the flow splitter in one embodiment includes an adjustable fluid gate or flap therewithin.

In some embodiments, the flow splitter may include a nozzle for controlling fluid pressure into the outlet elements and thereby into the catheter lumen or lumens.

There may be provided a flow diffuser in one or more of the fluid outlet elements.

According to another aspect of the present invention, there is provided a catheter assembly including a multi-lumen catheter and fluid supply apparatus for supplying fluid to said multi-lumen catheter, the fluid supply apparatus including an inlet element for coupling to a supply of catheter fluid, a plurality of fluid outlet elements arranged to be each coupled to a respective lumen of the catheter and a flow splitter arranged between the inlet element and the outlet elements and able to split or divert flow of fluid from the inlet to at least one of the plurality of outlets.

### DESCRIPTION OF THE DRAWINGS

Embodiments of the present invention are described below, by way of example only, with reference to the accompanying drawings, in which:

FIG. 1 is a schematic view in perspective of an embodiment of catheter assembly suitable for injecting contrast media into a patient;

FIGS. 2 and 3 are cross-sectional views of an example of multi-lumen catheter suitable for the assembly of FIG. 1;

FIG. 4 shows in schematic form in axial cross-section one embodiment of flow splitter;

FIG. 5 shows in schematic form in axial cross-section one embodiment of nozzle arrangement for the flow splitter of FIG. 1; and

FIG. 6 shows in schematic form in axial cross-section one embodiment of diffuser for the flow splitter of FIG. 1.

### DESCRIPTION OF THE PREFERRED EMBODIMENT

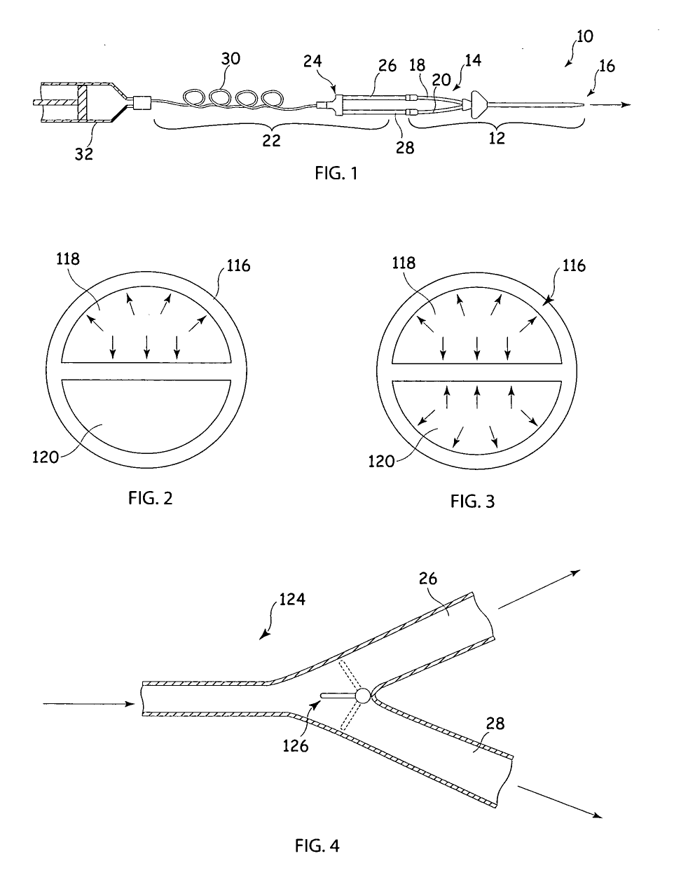

Referring to FIG. 1, there is shown in schematic form a preferred embodiment of catheter assembly 10 suitable for injecting contrast media into a patient, particularly for use in enhanced computer tomography (CT) scanning. It is to be understood that the teachings herein are not limited to CT scanning applications and could equally be applied to the injection of any fluid into a patient through a multi lumen catheter assembly.

The catheter assembly 10 of FIG. 1 includes a multi-lumen catheter 12 of conventional form and which includes at its proximal end 14 a plurality of feed tubes 18, 20 coupling to respective lumens (not shown in FIG. 1) of the catheter 12, in known manner. The lumens of the catheter 12 extend from its proximal end 14 to its distal end 16. At the distal end, the lumens are provided with apertures or ports (not shown) for feeding fluid, as well as diagnostic or introducer components through the distal end of the assembly 10 and thus into the treatment site in the patient.

The assembly 10 also includes a flow splitter or diverter unit 22 (hereinafter referred to as a flow splitter unit) for supplying fluid, such as contrast media, to the feed tubes 18, 20 and thus to a plurality of the lumens of the catheter 12.

The flow splitter unit 22 includes a splitter 24, described in further detail below, first and second fluid feed outlet tubes 26, 28, each coupled in fluid-tight manner to a respective one of the feed tubes 18, 20 of the catheter 16. A coiled extension tube 30 is coupled to the inlet of the splitter 24 and, at its proximal end, to a pressure injection unit 32, of any suitable nature known in the art, for supplying fluid to the assembly 12, 22. In this example, the injector 32 is filled with a contrast medium of any suitable type for enhancing CT scanning.

Referring now to FIG. 2, there is shown a cross-sectional view of an example of a catheter 116 having two internal lumens 118, 120. The upper lumen 118, as viewed in FIG. 3, is shown filled with a fluid dye, the arrows in the Figure depicting the outwardly directed pressure produced on the inner walls of the lumen 118 as a result of the pressure of the fluid. The lower lumen 120, as viewed in FIG. 2, is shown empty. It will be appreciated that in order to increase the volume of fluid which is fed through the catheter 116 in the manner shown in FIG. 2, the fluid pressure must be increased, resulting in an increasing pressure on the walls of the lumen 118. There is a physical limit to this pressure, beyond which there is the risk of damage to the lumen walls, to the couplings between the lumens and the various tubes of the assembly and/or to the distal end 16 of the catheter 116. Moreover, the pressurisation of the upper lumen 118 causes asymmetric stiffening and possibly distortion of the catheter 116, which in some circumstances can be disadvantageous.

Referring now to FIG. 3, the catheter 116 is shown with both lumens 118, 120 filled with fluid. As both lumens 118, 120 are in this view substantially equally filled with fluid, there is substantially even pressure in both halves (top and bottom as viewed in FIG. 3) of the catheter 116. Furthermore, with an effective doubling of the cross-sectional area of lumen through which fluid can pass, it is possible to increase the amount of fluid passing through the catheter without increasing pressure in the catheter. For enhanced CT scanning in particular this can mean increased supply of contrast media to the site to be diagnosed and thus improved scans.

FIG. 4 shows an embodiment of flow splitter 124 for use in the splitter unit 24 of FIG. 1. In this embodiment, the flow splitter 124 includes a gate 126 which can be pivoted between the three positions shown in the Figure, that is from the central position shown in full, whereupon fluid can pass to both of the outlet tubes 26 and 28, to one of the positions shown in dotted outline, to close-off one of the outlet tubes 26 and 28. A suitable control handle (not shown but of a design readily apparent to the skilled person) may be provided on the outside of the splitter unit 124 for manipulation by a surgeon or other clinician. Other types of control device for moving the gate 126 will be apparent to the skilled person.

The ability to control the flow into the tubes 26, 28 and thus into the lumens 118, 120 of the catheter can provide several advantages, including increased flow rate when fluid is fed to both lumens and differential stiffening/deflection of the catheter by feeding fluid to one of the lumens 118, 120, to assist for example in positioning or trackability of the catheter during deployment. Flow through a single lumen 118,120 may also be desired when lower flow rates are required for a particular diagnostic procedure.

Referring now to FIG. 5, there is shown an example of nozzle 130 which in this embodiment is a constriction flow splitter 224. The nozzle has the effect of increasing flow into the outlet tubes 26, 28 and of reducing the pressure into these.

Although FIG. 5 does not show a gate 126, such a gate may be provided in order to control where the fluid is channelled.

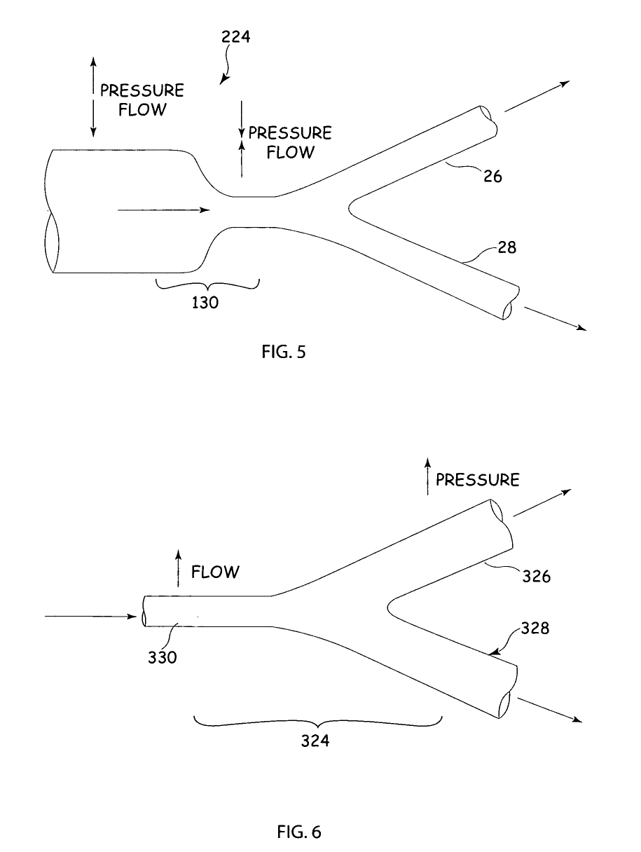

FIG. 6 shows an example of diffuser 324, which in this embodiment is in the form of outlet tubes 326, 328 of wider cross-sectional area compared to the inlet part 330 of the splitter. This has the effect of increasing the pressure in the outlet tubes 326, 328, as will be apparent to the skilled person.

The features of the gate 126 of FIG. 4, the nozzle of FIG. 5 and the diffuser of FIG. 6 can be used together in any combination of the three considered advantageous for a particular application or use.

What is claimed is:

1. Fluid supply apparatus for supplying fluid to a multi-lumen catheter including an inlet element for coupling to a supply of catheter fluid, a plurality of fluid outlet elements and a flow splitter arranged between the inlet element and the outlet elements and able to split or divert flow of fluid from the inlet to at least one of the plurality of outlets.

2. Fluid supply apparatus according to claim 1, wherein the flow splitter is configurable so as to adjust the flow to the outlet elements.

3. Fluid supply apparatus according to claim 2, wherein the flow splitter is settable to direct fluid to only one of the outlet elements or to a plurality thereof.

4. Fluid supply apparatus according to claim 2, wherein the flow splitter includes an adjustable fluid gate.

5. Fluid supply apparatus according to claim 1, wherein the flow splitter includes a nozzle.

6. Fluid supply apparatus according to claim 1, wherein the flow splitter includes a diffuser.

7. Fluid supply apparatus according to claim 1, wherein the flow splitter includes a diffuser in one or more of the fluid outlet elements.

8. A catheter assembly including a multi-lumen catheter and fluid supply apparatus for supplying fluid to said multi-lumen catheter, the fluid supply apparatus including an inlet element for coupling to a supply of catheter fluid, a plurality of fluid outlet elements arranged to be each coupled to a respective lumen of the catheter and a flow splitter arranged between the inlet element and the outlet elements and able to split or divert flow of fluid from the inlet to at least one of the plurality of outlets.

9. Fluid supply apparatus for supplying fluid to a multi-lumen catheter including an inlet element for coupling to a supply of catheter fluid, said inlet element being in the form of a coiled extension tube; a plurality of fluid outlet elements; and a flow splitter arranged between the inlet element and the outlet elements and able to split or divert flow of fluid from the inlet to at least one of the plurality of outlets; wherein the flow splitter includes an adjustable fluid gate settable in at least one first configuration to direct fluid to only one of the outlet elements and in a second configuration to direct fluid to said plurality of the outlet.
